(19) United States
(12) Patent Application Publication
(10) Pub. No.: US 2024/0389891 A1
(43) Pub. Date: Nov. 28, 2024

(54) ELECTROOSMOTIC PERFUSION WITH EXTERNAL MICRODIALYSIS PROBE

(71) Applicant: University of Pittsburgh - Of the Commonwealth System of Higher Education, Pittsburgh, PA (US)

(72) Inventors: Stephen G. Weber, Allison Park, PA (US); Jun Chen, Wexford, PA (US); Michael Trey Rerick, Reading, PA (US)

(73) Assignee: University of Pittsburgh - Of the Commonwealth System of Higher Education, Pittsburgh, PA (US)

(21) Appl. No.: 18/692,786

(22) PCT Filed: Sep. 16, 2022
(86) PCT No.: PCT/US2022/043896

§ 371 Date: Mar. 15, 2024

Related U.S. Application Data
(60) Provisional application No. 63/245,700, filed on Sep. 17, 2021.

(51) Int. Cl. A61B 5/145

(52) U.S. Cl. CPC A61B 5/14528; A61B 5/14546

(57) ABSTRACT

A probe can be situated within a tissue to perfuse one or more materials, collect material in response, and pass the collected material to a microdialysis probe situated external to the tissue based on fluid flow by electroosmosis. Perfusion and collection can be via orifices that are offset laterally and along a tissue depth. Variation of perfusion by varying materials perfused or rate of perfusion can be used to determine perfusion rates or other time-dependencies., concentrations of chemical species and rates of reactions of chemical species in the tissue.

### CROSS-REFERENCE TO RELATED APPLICATIONS

This application claims the benefit of U.S. Provisional Application 63/245,700, filed Sep. 17, 2021, which is incorporated herein by reference.

### ACKNOWLEDGMENT OF GOVERNMENT SUPPORT

This invention was made with government support under grant number GM044842 awarded by National Institutes of Health. The government has certain rights in the invention.

### FIELD

The disclosure pertains to electroosmotic probes.

### BACKGROUND

Assessing enzymatic activity quantitatively in vivo is challenging due to the lack of approaches available when using natural substrates. For example, studying ectopeptidase activity using natural substrates requires the ability to sample from the extracellular space of a living animal and quantify extracellular concentrations of peptide substrates and hydrolysis products in the sample. Sampling methods commonly utilized for probing the extracellular environment include push-pull perfusion (PPP) which can offer high spatial resolution and control of tissue residence time., but when online analysis is desired to reduce solute loss due to sample handling, the low nanoliter-per-minute flow rates associated with PPP are difficult to manage. Microdialysis is well-suited for online analysis with flow rates typically in the low microliter-per-minute range or lower. But assessing enzymatic activity has been carried out by introducing natural substrate to the tissue by passive diffusion through the microdialysis membrane into the tissue. Thus, microdialysis lacks control of residence time and substrate concentration. To obtain a rate requires information about a change in concentration over a certain time. This lack of control of the residence time in microdialysis makes it unsuitable for assessing enzymatic activity in vivo quantitatively. Alternative approaches are needed.

### SUMMARY

Electroosmotic (EO) probes comprise a probe body that defines probe cavity and a tapered probe tip terminating the probe body, wherein the probe body defines an orifice adapted to dispense a perfusate. A microdialysis (MD) probe is situated to receive a collection fluid responsive to dispensation of the perfusate from the orifice. In some examples, at least a first capillary is situated in the probe cavity and defines a flow channel and can extend beyond the probe body at a proximal end of the probe body. In representative embodiments, the probe cavity is defined in a direct laser writing material. A first electrode and a second electrode can be coupled to produce an electro-osmotic (EO) flow from the probe cavity to the MD probe, the EO flow created by at least one current source which can be coupled to the first and second electrodes and adapted to vary dispensation of the perfusate based on a variable current. The probe body and the MD probe can be secured to each other. Typically, the orifice of the probe body faces the MD probe.

In other examples, electroosmotic (EO) probes comprise a probe body that defines a first probe cavity and a second probe cavity, the first probe cavity defining a first orifice adapted to dispense a perfusate and the second probe cavity defining a second orifice situated to receive a collection fluid responsive to the dispensation of the perfusate from the first orifice. A first tapered probe tip terminates the first probe cavity and a second tapered probe tip terminates the second probe cavity. In some examples, the first probe cavity and the first tapered probe tip and the second probe cavity and the second tapered probe tip extend parallel to a common axis and the first probe tip and the second probe tip are displaced laterally is a direction perpendicular to the common axis. In some embodiments, the first probe cavity defines a first reservoir and a first flow channel coupled to communicate the perfusate to the first orifice and the second probe cavity defines a second flow channel coupled to receive the collection fluid from the second orifice. Typically, the second tapered probe tip is more distal along the common axis than the first tapered probe tip. In some examples, a first capillary is situated in the first probe cavity and defines the first flow channel and a second capillary is situated in the second probe cavity and defines the second flow channel. In representative examples, the first capillary and the second capillary extend to be proximate the first tapered probe tip and the second tapered probe tip, respectively. In typical embodiments, the first orifice is displaced along the common axis from the second orifice and the first orifice is situated to face toward the second flow channel and the second orifice is situated to face toward the first flow channel.

In further examples, the first probe cavity defines the first reservoir and a second reservoir, and the first and second reservoirs are coupled to the first flow channel to communicate respective perfusates. A first electrode can be coupled to the first reservoir and a second electrode can be coupled to the second reservoir. In some examples, the probe body defines a microdialysis (MD) cavity fluidically coupled to the second probe tip, the MD cavity defining a collection inlet and a collection outlet, and further comprising a microdialysis probe situated in the MD cavity. In representative examples, the MD cavity includes a dialysis fluid inlet at a proximal end of the MD cavity and a dialysis fluid outlet at a distal end of the MD cavity. The MD cavity generally can include a flow channel defined in a wall of the MD cavity and extending along a length of the MD cavity. The probe body can also define a waste cavity coupled to the flow channel in the MD cavity.

In some specific examples, a first electrode is coupled to the first probe tip and a second electrode is coupled to the MD cavity and are situated to produce an electro-osmotic (EO) flow from the first probe cavity to the MD membrane through the second probe cavity. A pump is arranged to produce an input flow to and an output flow from the MD cavity, the output flow capturing a portion of collection fluid passed through the MD membrane, wherein the second electrode is situated to direct the EO flow to the pump. In some examples, a first electrode and a second electrode are coupled to the first reservoir and the second reservoir and adapted to produce an electro-osmotic (EO) flow from the first reservoir, the second reservoir, or both the first and the second reservoirs to the MD membrane. At least one current source can be coupled to the first electrode and the second electrode to selectively dispense diffusate from one or both of the first reservoir and the second reservoir.

Methods comprise inserting a first probe and a second probe into a tissue, wherein the first probe is fixed with respect to the second probe, electro-osmotically providing a perfusate to the tissue from the first probe and collecting a fluid from the tissue in response to the perfusate with the second probe. The first probe and the second probe can be defined in a common probe body and are coupled to a first probe tip and a second probe tip, respectively, the common probe body further defining a cavity that is adapted to retain an MD probe so that the collected fluid is directed from the second probe to the MD probe. In some examples, the MD probe is situated internally with respect to the tissue. In typical examples, the method includes varying a current that electro-osmotically provides the perfusate to the tissue. In additional examples, the method includes coupling a first reservoir associated with a first perfusate and a second reservoir associated with a second perfusate to the first probe and selectively coupling the first and/or second perfusates to the tissue based on respective currents associated with the first and second reservoirs. The first perfusate and the second perfusate can be different and comprise one or more of a substrate and an inhibitor associated with activity of selected enzyme.

Methods can further includes coupling a first reservoir associated with a first perfusate, a second reservoir associated with a second perfusate, and a third reservoir associated with a third perfusate to the first probe and selectively coupling the first, second, and/or third perfusates or any combination thereof to the tissue based on respective currents associated with the first, second, and third reservoirs. In some examples, the first perfusate is a substrate, the second perfusate is an inhibitor, and the third perfusate is neither a substrate nor an inhibitor, where the substrate and the inhibitor are associated with activity of selected enzyme.

The foregoing and other objects, features, and advantages of the disclosed technology will become more apparent from the following detailed description, which proceeds with reference to the accompanying figures.

### BRIEF DESCRIPTION OF THE DRAWINGS

FIG. 1 illustrates a representative electroosmotic perfusion-internal microdialysis (EOP-IMD) device.

FIG. 1A is photograph of a representative device such as shown in FIG. 1.

FIG. 2 illustrates a system that uses a device such as shown in FIG. 1.

FIG. 3 illustrates a representative unitary electroosmotic perfusion-external microdialysis (EOP-EMD) device that includes laterally and vertically offset perfusion and collection orifices.

FIG. 4 illustrates a representative unitary EOP-EMD device that includes cavities for a plurality of perfusates.

FIGS. 5, 5B, and 5D illustrate a representative unitary EOP-EMD device that is formed by direct laser writing.

FIGS. 5A and 5C are sectional views of FIGS. 5 and 5B, respectively.

FIGS. 5E-5F are photographs of representative devices such as shown in FIGS. 5-5D.

FIG. 6 illustrates a representative microdialysis (MD) probe.

FIG. 7 illustrates a representative method.

FIG. 8 illustrates an EO probe system having flow channels defined on a planar substrate.

### DETAILED DESCRIPTION

### Introduction

The disclosure pertains to microdialysis measurements, methods, and apparatus based on first and second cannula (or a single cannula in some examples) and an electrical current (electroosmosis) for infusion of a substance into a tissue for which microdialysis measurement are made. One or more infusates can be used. and infusate concentration can be varied using an applied electrical current. The infusion current is directed into tissue through an orifice in a small, pointed probe tip and through the tissue, then into a microdialysis probe or into an orifice of a second small, pointed probe tip, and out of the tissue to a microdialysis probe or other external separation or analytical system. The first and second probe tips are arranged to reduce tissue trauma, decrease time required to reach steady state after changing the applied electrical current (compared to a microdialysis probe within the tissue), and direct perfusate and collect samples from tissue regions that tend to exhibit reduced trauma and thus can be more indicative of tissue response. In some examples, a single probe tip is used with a microdialysis probe that is situated completely or partially within a tissue under investigation. While the examples are described with reference to microdialysis of samples, samples can be treated in other ways or coupled directly for analysis.

### Terminology and General Considerations

As used herein, “probe” refers to a tube or other conduit defining a cavity for transporting fluids to and from a sample. A probe can include a flow channel, a tapered probe tip, and/or one or more fluid reservoirs. One or more probes can be defined in a common substrate so that, for examples, probes that introduce into and receive materials from a sample are secured to each other. In some examples, probes generally have cylindrical cross-sections but can have polygonal, elliptical, arcuate, curved, oval, or other cross-sections or combinations thereof. Probes generally terminate at a probe tip and the probe tip is typically conical or otherwise tapering. An orifice can be defined in the probe, typically in a distal portion of the flow channel to permit introduction of perfusates or to collect materials from a specimen. Particular probes and associated parts can be referred to as perfusion probes, perfusion tips, perfusion channels, and perfusion reservoirs for convenient illustration. Such probes can also generally serve as collection probes.

As noted above, orifices for passage of materials into and out of a sample are generally defined at untapered side wall portions of tubes, conduits, or any type of flow channel. Materials of interest can be directed into or collected from a sample via such orifices. In some configurations, a tapered probe tip is provided to facilitate insertion into a tissue and a side wall orifice is typically proximate the tapered portion so that the orifice is situated suitably deep within the tissue. The introduction of materials of interest is referred to herein as infusion or perfusion and the materials of interest are referred to infusates or perfusates. Probes having overall dimensions less than 10, 5, 2, or 1 mm are referred to as compact probes. For systems that include multiple probes, probe flow channel cross-sectional dimensions are between about 0.5 mm and 5 mm. Multiple probe tips are generally configured to have total separations of at least 5, 10, 20, 50, 100, 250, 500, or 1000 μm. Typically, a perfusion (or infusate) probe tip and a collection probed tip are offset laterally and vertically in use, with the collection probe tip typically more deeply inserted into a sample such as a tissue.

For convenience, fluids provided to a tissue or other sample from a probe in response to an electrical current or voltage are referred to herein as perfusates or infusates. Fluids received by a probe in response to perfusates are referred to herein as collected fluids or collected materials. Such fluids can include perfusates, materials responsive to perfusates, and specimen constituents that are independent of the perfusates. Fluids supplied to produce dialysis are referred to as dialysis fluids, and fluid outputs produced by dialysis is referred to as processed fluids or dialyzed fluids. Typically, processed fluids comprise a dialysis fluid with constituents obtained from a collected fluid by dialysis. Electrodes used to produce electroosmotic (EO) flows are generally coupled via one or more flow channels and can be situated upstream or downstream with respect to such flows. For example, electrodes can be situated at or in perfusate supplies, at dialysis flows, or other location.

Probe channels, fluid cavities, tubes, and other conduits for fluid flow can be defined using rigid glass or silica capillaries or formed of other materials using, for example, laser direct writing. In some examples, a laser system is used to produce a support that defines suitable cavities into which glass or silica tubes such as capillary tubes can be inserted. Electroosmotic flows are described as responsive to applied currents but applied voltages can be used as well. Fluids are referred to herein as conducted by channels that can be defined in glass, silica, plastics, or other materials. Channel cross-section are typically circularly, but oval, polygonal, or other shapes and combinations of such shapes can be used. Flow generally refers to electroosmotic flow, and not pump-based flow but dialysis fluids are typically pump-driven.

Direct laser writing materials are materials suitable for multiphoton lithography and include acrylates, acrylic hybrid organic/inorganic resists, Zr containing hybrid photopolymers such as SZ2080 and epoxy resins such as those identified as SU-8, SCR-701, and SCR-500. Such materials are generally used in two-photon polymerization processes.

In some examples, probe tips are referred to as perfusion tips or collection tips for convenient explanation. Flow channels and reservoirs are also referred to in some examples as associated with perfusion or collection to facilitate explanation. In examples, probes comprise a tapered tip and an associated flow channel.

As used in this application and in the claims, the singular forms “a,” “an,” and “the” include the plural forms unless the context clearly dictates otherwise. Additionally, the term “includes” means “comprises.” Further, the term “coupled” does not exclude the presence of intermediate elements between the coupled items. As used herein, “coupled” refers to electrical connections provided by electrically conductive paths as well as fluid connections that provide flow paths. In some cases, “coupled” refers to a connection of mechanical parts. It will be apparent from context, which type of coupling is used.

The systems, apparatus, and methods described herein should not be construed as limiting in any way. Instead, the present disclosure is directed toward all novel and non-obvious features and aspects of the various disclosed embodiments, alone and in various combinations and sub-combinations with one another. The disclosed systems, methods, and apparatus are not limited to any specific aspect or feature or combinations thereof, nor do the disclosed systems, methods, and apparatus require that any one or more specific advantages be present or problems be solved. Any theories of operation are to facilitate explanation, but the disclosed systems, methods, and apparatus are not limited to such theories of operation.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed systems, methods, and apparatus can be used in conjunction with other systems, methods, and apparatus. Additionally, the description sometimes uses terms like “produce” and “provide” to describe the disclosed methods. These terms are high-level abstractions of the actual operations that are performed. The actual operations that correspond to these terms will vary depending on the particular implementation and are readily discernible by one of ordinary skill in the art.

In some examples, values, procedures, or apparatus' are referred to as “lowest”, “best”, “minimum,” or the like. It will be appreciated that such descriptions are intended to indicate that a selection among many used functional alternatives can be made, and such selections need not be better, smaller, or otherwise preferable to other selections.

Examples are described with reference to directions indicated as “above,” “below,” “upper,” “lower,” and the like. These terms are used for convenient description, but do not imply any particular spatial orientation.

### Example 1

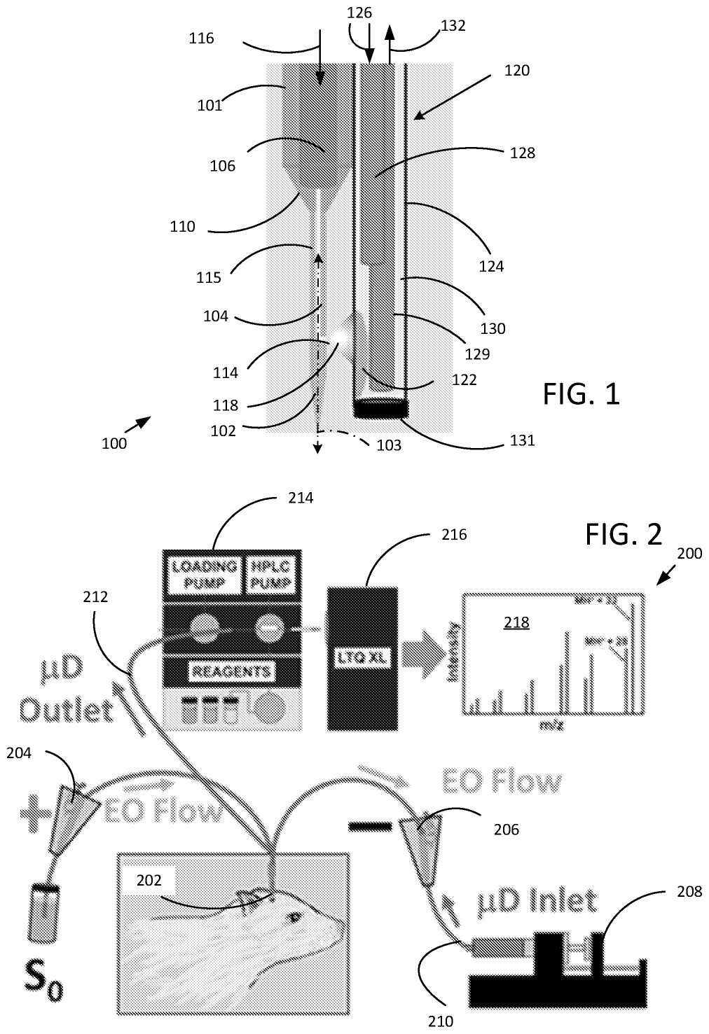

Referring to FIG. 1, a representative electroosmotic perfusion-internal microdialysis (EOP-IMD) device 100 includes a probe body 101 that defines a perfusion tip 102 that is coupled via a perfusion channel 104 to a first reservoir 106. The probe body 101 includes a tapered region 110 between the perfusion channel 104 and the first reservoir 106. The first reservoir 106 is generally defined by one or more glass or silica capillaries that are inserted into the probe body 101 but are not shown in FIG. 1. An orifice 114 is defined in the probe body 101 and thus permits an EO flow 116 into the EOP-IMD device 100 to exit the EOP-IMD device 100 as shown at 118 into a tissue or other sample of interest. As shown in FIG. 1, the orifice 114 need not be at the most distal portion of the perfusion tip 102 and can face away from an axis 103 of the perfusion tip 102. Typically, the orifice 114 is defined in a wall 115 of the perfusion channel 104. For convenience, a tip in combination with an associated flow channel can be referred to as a probe so that the perfusion tip 102 and the perfusion channel 104 form a perfusion probe. In other examples, collection probes similarly include a tip for insertion and an associated flow channel.

The EOP-IMD device 100 also includes a microdialysis (MD) probe 120 that is situated to receive perfusate, products associated with the perfusate, portions of tissue fluids, or other materials (referred to herein as collected materials) as indicated at 122. A MD membrane 124 is situated to receive the collected materials. In this example, the MD membrane 124 and a cap 131 define a cavity 130 for flow of a dialysis fluid into and out of the MD probe 120 via capillaries 128, 129. An exit flow 132 from within a volume defined by the membrane 130 generally includes any materials of interest (i.e., portions of the collected materials) for delivery to analytical equipment such as chromatography systems such as an HPLC or other system.

Perfusion from the perfusion probe tip 102 into the sample and through the MD membrane 130 can be generally controlled by a suitable current applied with electrodes coupled to fluids in the perfusion tip 102 and to fluids associated with a flow 132. These electrodes are omitted from FIG. 1 for convenient illustration. Dialyzer pumping with, for example, a syringe pump, produces a flow input 126 and the flow output 132 so that portions of the collected materials 122 are swept into the flow 132. Typically, electroosmotic flow electrodes are situated to urge the collected materials 122 toward the MD probe 120 and a pump is used to produce a flow in the MD probe 120 which captures portion of the collected materials 120.

FIG. 1A illustrates a representative EOP-IMD device that is to be situated internally in a tissue 150 that includes a probe having a tapered probe tip 152 that terminates a flow channel 154 and an MD probe 160.

### Example 2

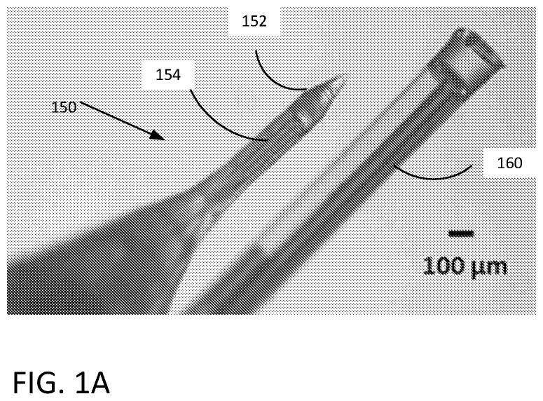

Referring to FIG. 2, a representative system 200 for in vivo brain measurement includes an EOP-IMD device 202 that includes a perfusion tip, perfusion channel, and an MD probe such as illustrated in FIG. 1. Flow of a substrate S₀ into the perfusion tip and a flow out of the MD probe is produced with a current between electrodes 204, 206. As shown in FIG. 2, this flow is referred to as an “EO flow.” A pump 208 such as a syringe pump is coupled to pump a dialysis fluid into the MD probe of the EOP-IMD device from an inlet 210 (referred to as a μD inlet and situated similarly to the flow associated with capillary 129 of FIG. 1) to an outlet 212 (referred to as a μD outlet and situated similarly to the flow input of FIG. 1) that provides dialyzed collection materials responsive to the EO flow from the probe tip along with the dialysis fluid. The outlet 212 is coupled to an HPLC system 214 which is in turn coupled to a linear ion trap mass spectrometer 216. A resulting chromatogram with mass spectra 218 can be presented on a display device and/or stored in one or more files in a computer-readable storage device.

### Example 3

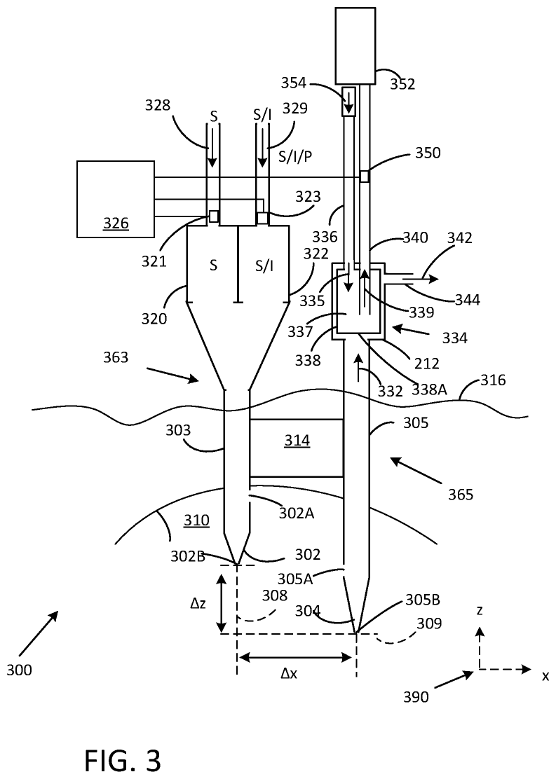

In the examples of FIGS. 1-2, a microdialysis probe is inserted into a tissue or other specimen of interest. Such insertion can produce tissue damage that can render resulting analysis unrepresentative, Referring to FIG. 3, a representative EOP external MD (EOP-EMD) probe 300 includes a perfusion probe 363 that includes a first tip 302 (a perfusion tip) that is fluidically coupled to a first flow channel 303 (a perfusion flow channel) and a collection probe that includes a second tip 304 (a collection tip) that is fluidically coupled to second flow channel 305 (a collection channel) that are displaced along an insertion axis 308 so that the collection tip 304 extends further into a tissue 310 being probed than the perfusion tip 302. The perfusion channel 303 and the collection channel 305 are separated by and fixed together with a support 314 that fixes their separation and prevents tip bending during manufacturing and tissue implantation. The perfusion tip 302 and the collection tip 304 are generally tapered to be narrowest at a most distal end to aid in tissue insertion and reduce tissue damage. As shown in FIG. 3, the perfusion tip 302, the collection tip 304, and portions of the perfusion channel 303 and the collection channel 305 penetrate a tissue surface 316.

The perfusion channel 303 is coupled to a first fluid reservoir 320 and a second fluid reservoir 322 (shown as providing substrate(S) and substrate and inhibitor (S/I) from which selected materials can be directed to the perfusion channel 302 with respective electrodes 321, 323 based on current from a power supply 326. Perfusate materials can be supplied with input flow channels 328, 329. Perfusate can be delivered to the tissue from orifices such as an orifice 302A situated in a side wall of the flow channel 303 or an orifice 302B situated at a distal end of the perfusion tip 302.

The collection flow channel 305 is coupled to direct a collected flow 332 to a microdialysis probe 334 situated external to the tissue 310. The microdialysis probe 334 includes a membrane 338 coupled to receive a dialysis fluid flow 335 produced by a pump 354 at an input 336 and deliver the dialysis fluid flow 335 to a volume 337 defined by the membrane 338. Typically, the membrane 338 is constructed from a tube whose sides are formed of a membrane material. An end surface 338A of such a tube need not be a membrane material.

The collected flow 332 can be received via orifices such as an orifice 305A in a side wall associated with the flow channel 305 or an orifice 305B situated at a distal end of the collection tip 304. One or more such orifices can be used as convenient and different locations of orifices associated with the perfusion channel 303 and the collection channel 305 can be used. In the example of FIG. 3, the perfusion tip 302 and the collection tip 304 have a separation Δz along a z-axis and a lateral separation Δx along an x-axis of a coordinate system 390. An output flow 339 based on portions of the collected flow 332 coupled through the membrane 338 and the dialysis fluid is provided to an output 340. A remainder 342 of the collected flow 332 is delivered to a waste output 344. The output flow 339 can be delivered to an analysis system 352 such as an HPLC system.

In the example of FIG. 3, orifices for perfusion and collection are offset laterally and vertically in the tissue 310, wherein the collection tip 304 is more distally located. Other arrangements can be used and Δx and Δz are generally range from 0 to 1 mm, 0.1 to 0.5 mm, 0.2 to 0.5 mm, 0.1 to 0.25 mm. As noted above, perfusion and collection orifices can face along a common axis (in this example, facing in −x and +x directions) with or without a vertical (z-axis) offset. In other examples, orifices are situated at tip ends.

The EOP-EMD probe 300 defines the flow channels for collection and delivery in a unitary arrangement, wherein both are defined in a common, connected material and are fixed to each other with the support 314 that can be defined in the same common, connected material. In one example, two-photon polymerization is used with a suitable material. In some examples, the perfusion probe 363 (including reservoirs 320, 322), the collection probe 365 are defined as a single piece. The support 314 can be defined as part of this single piece, separately defined, or omitted

Perfusate composition and rate can be controlled with a variable current from the power source 326. Typically, a current is applied to control a flow rate from one or both of the electrodes 321, 323 to the electrode 350. The current can also be applied so that material in the first fluid reservoir 320 and/or the second fluid reservoir 322 is supplied to the tissue 310. In FIG. 3, the electrodes 321, 323 are typically situated distant from the perfusion probe 363 and the collection probe 365 by 1, 2, 5, 10 cm or more along the input flow channels 328, 329.

### Example 4

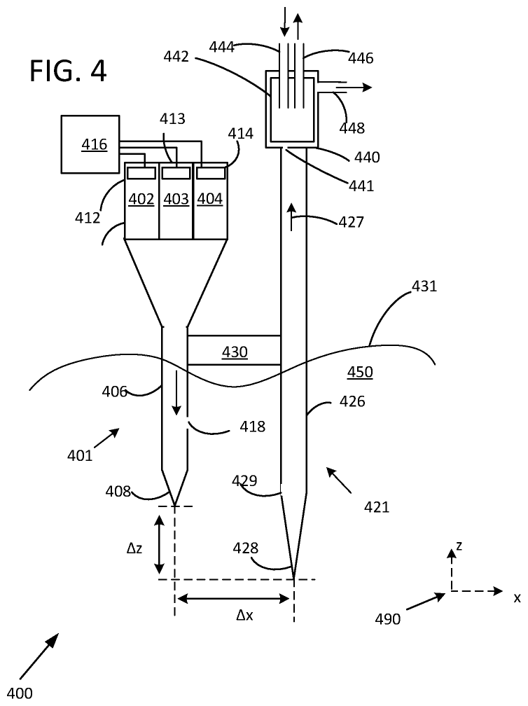

With reference to FIG. 4, an EOP-EMD probe 400 includes a perfusion probe 401 that includes first, second, and third reservoirs 402-404, a flow channel 406 (a perfusion flow channel), and a perfusion tip 408. The first, second, and third reservoirs 402-404 are coupled to the flow channel 406 that terminates at the flow tip 408. Each of the reservoirs 402-404 is associated with a respective electrode 412-414 so that materials from the reservoirs 402-404 can be supplied individually or in combination at an orifice 418 based on currents supplied from one or more power supplies 416. A collection probe 421 is coupled to the perfusion probe 401 with a support 430 and includes a flow channel 426 and a tip 428. An orifice 429 is situated to collect material from a tissue 450 in response to perfusate from the orifice 418. The flow channel 426 couples a collected flow 427 to a microdialysis (MD) probe 440 through an orifice 441 and includes a membrane 442, dialysis input and output channels 444, 446 and a waste outlet 448.

Flow channels, reservoirs, tips, and other flow cavities can be defined in a common material and fabricated as a single piece. In some cases, glass or silica capillaries or other rigid or flexible tubing are used to line cavities in the single piece and/or for coupling to remote material supplies or analytical instrumentation that can be distant from the tissue 450. The tips 408, 428 are generally conical or other shape that is pointed at a distal end for insertion into the tissue 450 through a tissue surface 431. In other examples, individual glass, silica, metals, or other materials are used. In the same manner as in the example of FIG. 3, the MD probe 440 is external to the tissue 450 and the tips 408, 428 are separated along both an x-direction and a y-direction as defined by a coordinate system 490.

### Example 5

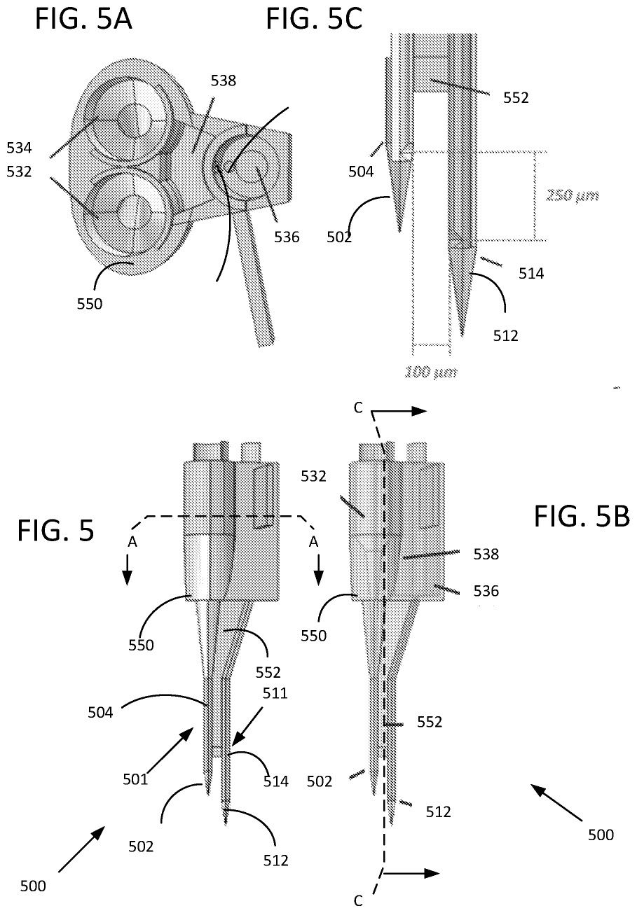

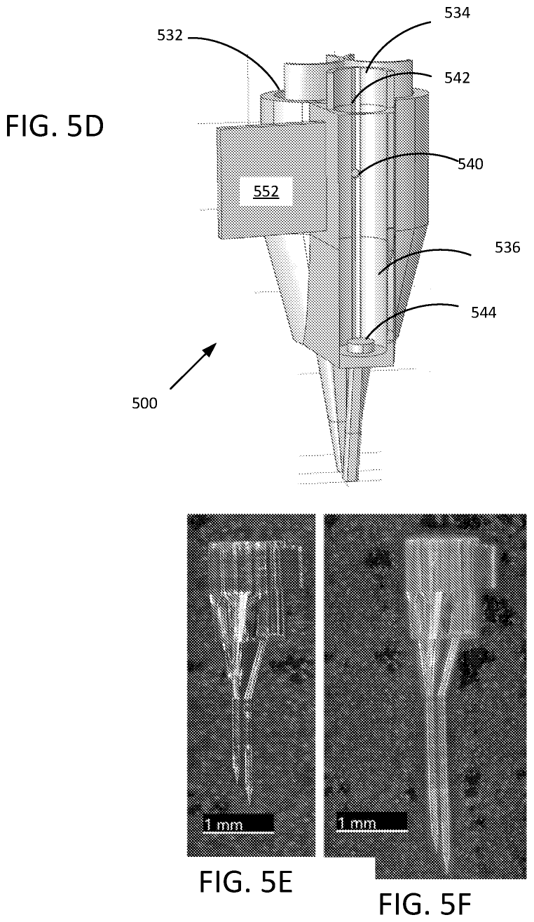

Referring to FIGS. 5 and 5A-5F, a representative unitary EOP-EMD device 500 defined in a substrate 550. The EOP-EMD device 500 includes a perfusion probe 501 that includes a perfusion tip 502 and a perfusion channel 504 and a collection probe 511 that includes a collection tip 512 and a collection channel 514. The perfusion channel 504 is coupled to first and second capillaries inserted into first and second cylindrical recesses 532, 534 that are defined in the substrate 550. The capillaries can be arranged to be wider at a top (i.e., more distant from the perfusion tip 502) and narrower at a bottom (i.e., closer to the perfusion tip and inserted into the substrate 550). Wider capillary portions are more robust and more fragile portions can be within and secured to the substrate 550. A support rib 552 secures the perfusion channel 504 and the collection channel 514. The collection channel 514 is coupled to an MD 536 cavity that is adapted to receive or form a part of an MD probe. In addition, a waste cavity 538 is coupled via an orifice 540 to the MD probe cavity 536 and includes a channel 542 that permits flow around an MD unit or MD membrane situated in the MD cavity 536. A pedestal 544 or other spacer is situated at a collection fluid input end of the MD cavity 536 and can serve to space an MD unit to permit flow of collected fluids from the collection channel 514 into the MD chamber 536 and around an MD unit situated in the MD chamber 536. Representative dimensions are shown in FIG. 5C and photographs of a representative device are shown in FIGS. 5E-5F.

### Example 6

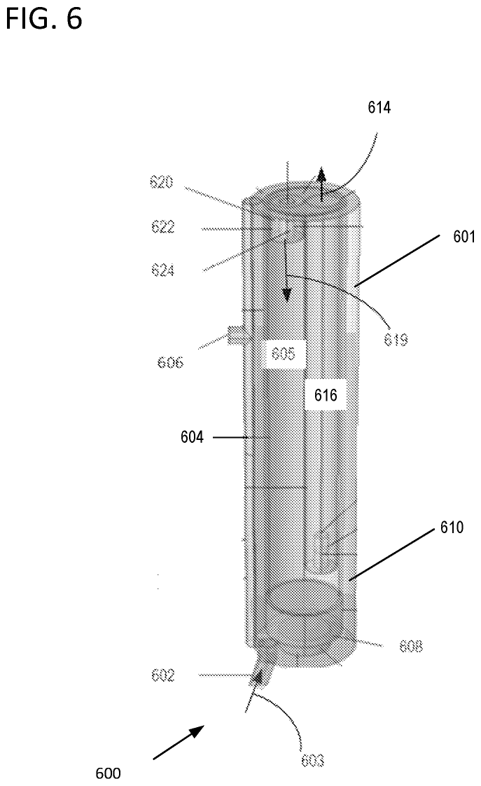

Referring to FIG. 6, a section of a representative EOP-EMD device 600 is shown coupled to a collection channel 602 that is adapted to deliver an input flow 603 of collected fluid to an MD sampling chamber defined by a support structure 601 that can be formed by direct laser writing. The collected fluid can flow about an MD membrane 604 and exit at an orifice 606. A glue plug 608 is situated for sealing so that the collection fluid and a dialysis fluid 619 do not physically mix. A shaded volume 610 prevents the microdialysis probe's solidified glue plug 608 from inhibiting flow 603 into the sampling chamber 601. The dialysis fluid 619 is introduced with an MD inlet capillary 620 into a volume defined by the MD membrane 604. The MD inlet capillary has an outer wall 622 and defines a flow channel 624. The dialysis fluid 619 and extracted portions of the collection fluid are delivered to an output 614 defined by a capillary 616.

### Example 7

In a representative example, a collection tip inlet is offset vertically by 250 μm from a distal end of the collection tip to maximize the volume of sampled tissue not in contact with a probe. The horizontal distance between a perfusion tip and the collection tip is 100 μm, corresponding to a distance of approximately 260 μm between the center of each orifice. The outer diameter of both the perfusion tip and the collection tip is 90 μm and both have 50 μm internal channel diameters but taper to be narrower and “pointy” toward respective tip ends. The tip support has a thickness of 10 μm, with the lower 0.1 mm forming a wedge to a final thickness of 1 μm. Conical portions terminating the perfusion tip and the collection tip are adapted to reduce or minimize penetration injury with an opening angle of 20°. The collection channel leads to a cylindrical microdialysis sampling chamber (height=1.35 mm, diameter=290 μm). This MD sampling chamber also connects to a waste reservoir (volume of about 88 μL). The overall dimensions of such a device can be 0.965 mm (W)×0.950 mm (L)×3.61 mm (H).

Such devices can be printed by 3D direct laser writing (Nanoscribe Photonic Professional, GT) with IP-S resist. Two-photon polymerization of the IP-S resist can used for printing by a femtosecond pulsed laser at 680 nm. Probe length can be modulated to, for example, permit probing of a selected brain region. FIGS. 5E-5F illustrate representative probes having 1.25 mm and 2.25 mm collection probe lengths, respectively. Probe length is generally used to establish probe penetration depth. The dimensions of the perfusion tip and the collection tip, the tip support, and tip entrance angle are generally selected to minimize the negative effects of penetration injury. As shown in some examples, rather than inserting the MD probe into tissue, the MD probe is housed externally within the MD sampling chamber. By applying a current to the perfusion inlet and grounding the MD inlet, electroosmotic flow is used to perfuse the tissue directionally toward the collection channel. The sampled solution travels through the collection channel to the MD sampling chamber. The volume within the MD sampling chamber is continuously filled with this solution, and collected analytes diffuse through the MD membrane for analysis.

### Example 8

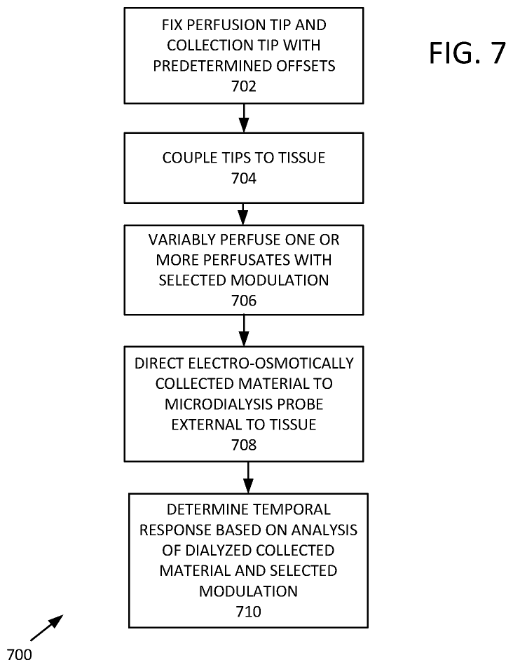

With reference to FIG. 7, a method 700 includes fixing a perfusion tip with respect to a collection tip at 702 so that perfusion and collection orifices are offset in two dimensions. At 704, the tips are coupled to tissue and at 706 one or more perfusates are electroosmotically directed into the tissue. At 708, collected material associated with the perfusate is directed to a microdialysis probe that is situated external to the tissue. At 710, the dialyzed collected material can be analyzed to, for example, determine a temporal response of the tissue based in variations or modulations of perfusate produce by varying drive currents. In representative examples, substrates and/or inhibitors associated with enzyme activity are introduced to study enzymatic activity in vivo.

### Example 9

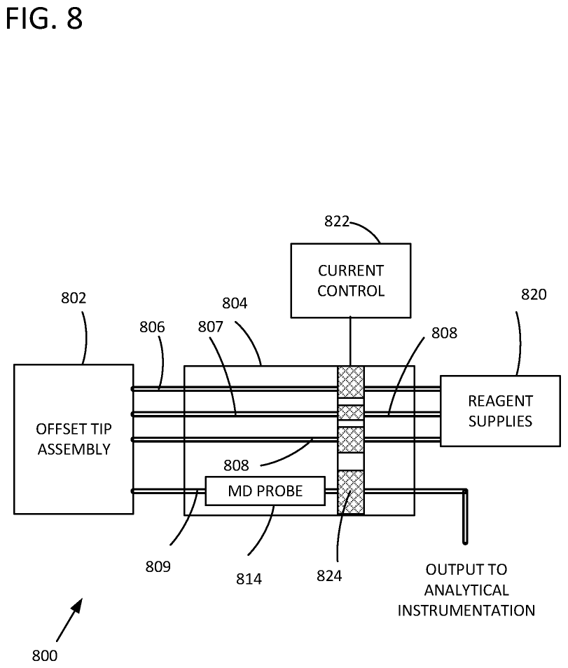

Referring to FIG. 8, a representative EOP-EMD probe system 800 includes an offset probe assembly 802 such as shown in FIG. 5A that is fluidically coupled to flow channels 806-809 defined on a substrate 804. In this example, flow channels 806-808 are used to couple reagents to the offset probe assembly 802 for infusion/perfusion and the flow channel 809 receives collected materials responsive to the infusion/perfusion for delivery to an MD probe 814. Reagents are supplied from sources 820 and electroosmotic currents are provided by a current control 822 that is coupled to a set of electrodes 824. Additional flow channels are provided for the MD probe 814 to provide dialysis fluid but are not shown in FIG. 8.

### ADDITIONAL REPRESENTATIVE EMBODIMENTS

Embodiment 1 is an electroosmotic (EO) probe, including: a probe body that a defines a first probe cavity and a second probe cavity, the first probe cavity defining a first orifice adapted to dispense a perfusate and the second probe cavity defining a second orifice situated to receive a collection fluid responsive to the dispensation of the perfusate from the first orifice; and a first tapered probe tip terminating the first probe cavity and a second tapered probe tip terminating the second probe cavity.
Embodiment 2 includes the subject matter of Embodiment 1, and further specifies that the first probe cavity and the first tapered probe tip and the second probe cavity and the second tapered probe tip extend parallel to a common axis and the first probe tip and the second probe tip are displaced laterally is a direction perpendicular to the common axis.
Embodiment 3 includes the subject matter of any of Embodiments 1-2, and further specifies that the first probe cavity defines a first reservoir and a first flow channel coupled to communicate the perfusate to the first orifice and the second probe cavity defines a second flow channel coupled to receive the collection fluid from the second orifice.
Embodiment 4 includes the subject matter of any of Embodiments 1-3, and further specifies that the second tapered probe tip is more distal along the common axis than the first tapered probe tip.
Embodiment 5 includes the subject matter of any of Embodiments 1-4, and further includes a first capillary situated in the first probe cavity and defining the first flow channel and a second capillary situated in the second probe cavity and defining the second flow channel.
Embodiment 6 includes the subject matter of any of Embodiments 1-5, and further specifies that the first capillary and the second capillary extend to be proximate the first tapered probe tip and the second tapered probe tip, respectively.
Embodiment 7 includes the subject matter of any of Embodiments 1-6 and further specifies that the first orifice is displaced along the common axis from the second orifice and the first orifice is situated to face toward the second flow channel.
Embodiment 8 includes the subject matter of any of Embodiments 1-7, and further specifies that the second orifice is situated to face toward the first flow channel.
Embodiment 9 includes the subject matter of any of Embodiments 1-8 and further specifies that the first probe cavity defines the first reservoir and a second reservoir, and the first and second reservoirs are coupled to the first flow channel to communicate respective perfusates.
Embodiment 10 includes the subject matter of any of Embodiments 1-9, and further includes a first electrode coupled to the first reservoir and a second electrode coupled to the second reservoir.
Embodiment 11 includes the subject matter of any of Embodiments 1-10, and further specifies that the probe body defines a microdialysis (MD) cavity fluidically coupled to the second probe tip, the MD cavity defining a collection inlet and a collection outlet, and further includes a dialysis membrane situated in the MD cavity.
Embodiment 12 includes the subject matter of any of Embodiments 11-1, and further specifies that the MD cavity includes a dialysis fluid inlet at a proximal end of the MD cavity and a dialysis fluid outlet at a distal end of the MD cavity.
Embodiment 13 includes the subject matter of any of Embodiments 1-12, and further specifies that the MD cavity includes a flow channel defined in a wall of the MD cavity and extending along a length of the MD cavity.
Embodiment 14 includes the subject matter of any of Embodiments 1-13, and further specifies that the probe body defines a waste cavity coupled to the flow channel in the MD cavity.
Embodiment 15 includes the subject matter of any of Embodiments 1-14, and further includes: a first electrode coupled to the first probe tip and a second electrode coupled to the MD cavity and situated to produce an electro-osmotic (EO) flow from the first probe cavity to the MD membrane through the second probe cavity; and a pump adapted to produce an input flow to and an output flow from the MD cavity, the output flow capturing a portion of collection fluid passed through the MD membrane, wherein the second electrode is situated to direct the EO flow to the pump.
Embodiment 16 includes the subject matter of any of Embodiments 1-15, and further includes a first electrode and a second electrode coupled to the first reservoir and the second reservoir and adapted to produce an electro-osmotic (EO) flow from the first reservoir, the second reservoir, or both the first and the second reservoirs to the MD membrane.
Embodiment 17-1 includes the subject matter of any of Embodiments 1-16, and further includes at least one current source coupled to the first electrode and the second electrode to selectively dispense diffusate from one or both of the first reservoir and the second reservoir.
Embodiment 17-2 includes the subject matter of any of Embodiments 1-16 and further includes at least one current source is coupled to the first electrode, the second electrode, and a third electrode to selectively dispense diffusate from some or all of the first reservoir, the second reservoir, and a third reservoir, respectively.
Embodiment 18 is a method, including: inserting a first probe and a second probe into a tissue, wherein the first probe is fixed with respect to the second probe; electro-osmotically providing a perfusate to the tissue from the first probe; and collecting a fluid from the tissue in response to the perfusate with the second probe.
Embodiment 19 includes the subject matter of Embodiment 18, further wherein the first probe and the second probe are defined in a common probe body and are coupled to a first probe tip and a second probe tip, respectively, the common probe body further defining a cavity that is adapted to retain an MD probe, and further includes directing the collected fluid from the second probe to the MD probe.
Embodiment 20 includes the subject matter of any of Embodiments 18-19, and further includes situating the MD probe internally with respect to the tissue.
Embodiment 21 includes the subject matter of any of Embodiments 18-20 and further includes varying a current that electro-osmotically provides the perfusate to the tissue.
Embodiment 22 includes the subject matter of any of Embodiments 18-21, and further includes coupling a first reservoir associated with a first perfusate and a second reservoir associated with a second perfusate to the first probe and selectively coupling the first and/or second perfusates to the tissue based on respective currents associated with the first and second reservoirs.
Embodiment 23 includes the subject matter of any of Embodiments 18-22, and further specifies that the first perfusate and the second perfusate are different and comprise one or more of a substrate and an inhibitor associated with activity of selected enzyme.
Embodiment 24 includes the subject matter of any of Embodiments 18-23, and further includes coupling a first reservoir associated with a first perfusate, a second reservoir associated with a second perfusate, and a third reservoir associated with a third perfusate to the first probe and selectively coupling the first, second, and/or third perfusates or any combination thereof to the tissue based on respective currents associated with the first, second, and third reservoirs.
Embodiment 25 includes the subject matter of any of Embodiments 18-24, and further specifies that the first perfusate is a substrate, the second perfusate is an inhibitor, and the third perfusate is neither a substrate nor an inhibitor, where the substrate and the inhibitor are associated with activity of selected enzyme.
Embodiment 26 is an electroosmotic (EO) probe, including: a probe body that defines probe cavity and a tapered probe tip terminating the probe body, wherein the probe body defines an orifice adapted to dispense a perfusate; and a microdialysis (MD) probe situated to receive a collection fluid responsive to dispensation of the perfusate from the orifice.
Embodiment 27 includes the subject matter of Embodiment 26, and further includes at least a first capillary situated in the probe cavity and defining a flow channel.
Embodiment 28 includes the subject matter of any of Embodiments 26-27, and further specifies that the first capillary extends beyond the probe body at a proximal end of the probe body.
Embodiment 29 includes the subject matter of any of Embodiments 26-28, and further specifies that the probe cavity is defined in a direct laser writing material.
Embodiment 30 includes the subject matter of any of Embodiments 26-29, and further includes a first electrode and a second electrode coupled to produce an electro-osmotic (EO) flow from the probe cavity to the MD probe.
Embodiment 31 includes the subject matter of any of Embodiments 26-30, and further includes at least one current source coupled to the first and second electrodes and adapted to vary dispensation of the perfusate based on a variable current.
Embodiment 32 includes the subject matter of any of Embodiments 26-31, and further specifies that the probe body and the MD probe are secured to each other.
Embodiment 33 includes the subject matter of any of Embodiments 26-32, and further specifies that the orifice of the probe body faces the MD probe.

In view of the many possible embodiments to which the principles of the disclosed technology may be applied, it should be recognized that the illustrated embodiments are only preferred examples and should not be taken as limiting the scope of the disclosure.

1. An electroosmotic (EO) probe, comprising:
a probe body that a defines a first probe cavity and a second probe cavity, the first probe cavity defining a first orifice adapted to dispense a perfusate and the second probe cavity defining a second orifice situated to receive a collection fluid responsive to the dispensation of the perfusate from the first orifice; and
a first tapered probe tip terminating the first probe cavity and a second tapered probe tip terminating the second probe cavity.

2. The EO probe of claim 1, wherein the first probe cavity and the first tapered probe tip and the second probe cavity and the second tapered probe tip extend parallel to a common axis and the first probe tip and the second probe tip are displaced laterally is a direction perpendicular to the common axis.

3. The EO probe of claim 2, wherein the first probe cavity defines a first reservoir and a first flow channel coupled to communicate the perfusate to the first orifice and the second probe cavity defines a second flow channel coupled to receive the collection fluid from the second orifice.

4. The EO probe of claim 3, wherein the second tapered probe tip is more distal along the common axis than the first tapered probe tip.

5. The EO probe of claim 3, further comprising a first capillary situated in the first probe cavity and defining the first flow channel and a second capillary situated in the second probe cavity and defining the second flow channel.

6. The EO probe of claim 5, wherein the first capillary and the second capillary extend to be proximate the first tapered probe tip and the second tapered probe tip, respectively.

7. The EO probe of claim 6, wherein the first orifice is displaced along the common axis from the second orifice and the first orifice is situated to face toward the second flow channel.

8. The EO probe of claim 7, wherein the second orifice is situated to face toward the first flow channel.

9. The EO probe of claim 3, wherein the first probe cavity defines the first reservoir and a second reservoir, and the first and second reservoirs are coupled to the first flow channel to communicate respective perfusates.

10. The EO probe of claim 9, further comprising a first electrode coupled to the first reservoir and a second electrode coupled to the second reservoir.

11. The EO probe of claim 1, wherein the probe body defines a microdialysis (MD) cavity fluidically coupled to the second probe tip, the MD cavity defining a collection inlet and a collection outlet, and further comprising a dialysis membrane situated in the MD cavity.

12. The EO probe of claim 11, wherein the MD cavity includes a dialysis fluid inlet at a proximal end of the MD cavity and a dialysis fluid outlet at a distal end of the MD cavity.

13. The EO probe of claim 12, wherein the MD cavity includes a flow channel defined in a wall of the MD cavity and extending along a length of the MD cavity.

14. The EO probe of claim 13, wherein the probe body defines a waste cavity coupled to the flow channel in the MD cavity.

15. The EO probe of claim 1, further comprising:
a first electrode coupled to the first probe tip and a second electrode coupled to the MD cavity and situated to produce an electro-osmotic (EO) flow from the first probe cavity to the MD membrane through the second probe cavity; and
a pump adapted to produce an input flow to and an output flow from the MD cavity, the output flow capturing a portion of collection fluid passed through the MD membrane, wherein the second electrode is situated to direct the EO flow to the pump.

16. The EO probe of claim 15, further comprising a first electrode and a second electrode coupled to the first reservoir and the second reservoir and adapted to produce an electro-osmotic (EO) flow from the first reservoir, the second reservoir, or both the first and the second reservoirs to the MD membrane.

17. The EO probe of claim 15, further comprising at least one current source coupled to the first electrode and the second electrode to selectively dispense from one or both of the first reservoir and the second reservoir.

18. A method, comprising:
inserting a first probe and a second probe into a tissue, wherein the first probe is fixed with respect to the second probe;
electro-osmotically providing a perfusate to the tissue from the first probe; and
collecting a fluid from the tissue in response to the perfusate with the second probe.

19. The method of claim 18, further wherein the first probe and the second probe are defined in a common probe body and are coupled to a first probe tip and a second probe tip, respectively, the common probe body further defining a cavity that is adapted to retain an MD probe, and further comprising directing the collected fluid from the second probe to the MD probe.

20. The method of claim 19, further comprising situating the MD probe internally with respect to the tissue.

21. The method of claim 18, further comprising varying a current that electro-osmotically provides the perfusate to the tissue.

22. The method of claim 18, further comprising coupling a first reservoir associated with a first perfusate and a second reservoir associated with a second perfusate to the first probe and selectively coupling the first and/or second perfusates to the tissue based on respective currents associated with the first and second reservoirs.

23. The method of claim 22, wherein the first perfusate and the second perfusate are different and comprise one or more of a substrate and an inhibitor associated with activity of selected enzyme.

24. The method of claim 18, further comprising coupling a first reservoir associated with a first perfusate, a second reservoir associated with a second perfusate, and a third reservoir associated with a third perfusate to the first probe and selectively coupling the first, second, and/or third perfusates or any combination thereof to the tissue based on respective currents associated with the first, second, and third reservoirs.

25. The method of claim 24, wherein the first perfusate is a substrate, the second perfusate is an inhibitor, and the third perfusate is neither a substrate nor an inhibitor, where the substrate and the inhibitor are associated with activity of selected enzyme.

26. An electroosmotic (EO) probe, comprising:
a probe body that defines probe cavity and a tapered probe tip terminating the probe body, wherein the probe body defines an orifice adapted to dispense a perfusate; and
a microdialysis (MD) probe situated to receive a collection fluid responsive to dispensation of the perfusate from the orifice.

27. The EO probe of claim 26, further comprising at least a first capillary situated in the probe cavity and defining a flow channel.

28. The EO probe of claim 27, wherein the first capillary extends beyond the probe body at a proximal end of the probe body.

29. The EO probe of claim 26, wherein the probe cavity is defined in a direct laser writing material.

30. The EO probe of claim 26, further comprising a first electrode and a second electrode coupled to produce an electro-osmotic (EO) flow from the probe cavity to the MD probe.

31. The EO probe of claim 30, further comprising at least one current source coupled to the first and second electrodes and adapted to vary dispensation of the perfusate based on a variable current.

32. The EO probe of claim 26, wherein the probe body and the MD probe are secured to each other.

33. The EO probe of claim 26, wherein the orifice of the probe body faces the MD probe.
